(19) United States
(12) Patent Application Publication
(10) Pub. No.: US 2024/0271174 A1
(43) Pub. Date: Aug. 15, 2024

(54) FGF21 PRODUCTION METHOD USING THERMOELASTICITY OF FGF21

(71) Applicant: TODD PHARM CO., LTD., Seoul (KR)

(72) Inventors: Sun-Shin CHA, Seoul (KR); Soo-Bong PARK, Seoul (KR); Ye-Eun JUNG, Seoul (KR); Da-Woon BAE, Seoul (KR); Bo-Gyeong JEONG, Seoul (KR); Bu-Gyeong KANG, Seoul (KR); Jung-hyun LEE, Busan (KR); Hyung-Soon YIM, Seoul (KR); Kyeong Won LEE, Busan (KR); Young Jun AN, Ansan (KR)

(21) Appl. No.: 18/035,087

(22) PCT Filed: Sep. 27, 2021
(86) PCT No.: PCT/KR2021/013090

§ 371 Date: May 2, 2023

(30) Foreign Application Priority Data
Nov. 3, 2020 (KR) 10-2020-0145101

(51) Int. Cl. C12P 21/02; C07K 14/50

(52) U.S. Cl. CPC C12P 21/02; C07K 14/50

(57) ABSTRACT

The present application relates to a method of producing or purifying an FGF21 protein by using the heat-elasticity of FGF21.

The method of producing or purifying the FGF21 protein according to an embodiment of the present application utilizes the heat stability and heat-elasticity of the FGF21 protein that is not denatured or precipitated even upon heat treatment and returns to the original structure when re-cooled after heat treatment, resulting in advantages of improving efficiency by simplifying a protein production process.

### CROSS-REFERENCE TO RELATED APPLICATIONS

This application is a U.S. national phase application filed under 35 U.S.C. § 371 of International Application No. PCT/KR2021/013090, filed Sep. 27, 2021, which is based upon and claims the benefit of priority from prior Korean Application No. 10-2020-0145101, filed Nov. 3, 2020.

### REFERENCE TO A SEQUENCE LISTING

The Sequence Listing associated with this application is filed in electronic format via EFS-Web and is hereby incorporated into the specification in its entirety. The name of the text file containing the Sequence Listing is “PX068197US_Seq”. The size of the text file is 5 KB, and the file was created on Nov. 30, 2023.

### TECHNICAL FIELD

The present application relates to a method of producing or purifying an FGF21 protein using heat-elasticity of FGF21.

### BACKGROUND ART

A mammalian-derived fibroblast growth factor (FGF) is a protein that regulates glucose/lipid metabolism, cell differentiation and proliferation, etc., by binding to an ectodomain of a receptor present in cell membrane and activating a tyrosine kinase domain of the receptor present in cytoplasm. The FGF family consists of 22 proteins that are structurally similar, and is divided into 7 subfamilies. FGFs of five subfamilies (i.e., FGF1, FGF4, FGF7, FGF8, and FGF9 subfamilies) require heparin or heparan sulfate to bind with FGFR. Heparin/heparan sulfate is a negatively charged polysaccharide, which belongs to the Glycosaminoglycan (GAG) family, interacts with a variety of proteins to regulate numerous functions on both the cell surface and extracellular matrix. FGFs exhibit high affinity for heparin/heparan sulfate, allowing them to function as paracrine that remain in close proximity to the cells that secrete them. FGF21 belongs to the endocrine subfamily (FGF19 subfamily) that includes FGF23 and FGF15/19. FGF21 is a major endogenous agonist of the FGF21 receptor, consisting of co-receptors FGF receptor 1c and β-Klotho.

FGF21 has recently been widely studied as an active ingredient in various therapeutic agents. In detail, in July 2019, Yuhan Corporation signed a license agreement with Boehringer Ingelheim, a multinational pharmaceutical company in Germany, for the purpose of developing a therapeutic agent for nonalcoholic steatohepatitis (NASH), and such a therapeutic agent is a dual agonist, in which FGF21 and glucagon-like peptide-1 (GLP-1) are fused. Currently, non-clinical toxicity test studies have been completed for the substance, and it is known that joint research is underway with the goal of clinical entry. In addition, MERCK, a multinational pharmaceutical company, is also developing MK-3655, a monoclonal antibody therapeutic agent that acts on the β-Klotho/FGFR1c composite, for the purpose of treatment of NASH. In this regard, it is known that FGF21 is a new drug candidate substance bringing attention in domestic and international pharmaceutical markets as a therapeutic agent for diabetes and NASH.

In addition, as various pharmacological effects on the brain, bone, pancreas, liver, etc. upon the FGF21 administration have been studied, the scope of application of a therapeutic agent including FGF21 as an active ingredient is expected to gradually expand.

Accordingly, the present inventors have made efforts to develop a method of efficiently purifying and producing the FGF21 protein, and as a result, it is confirmed that the FGF21 protein is not denatured even when a temperature higher than the general protein denaturation temperature is applied, and the physiological activity thereof is also maintained. Using this, a method of producing FGF21 protein is developed and the disclosure was completed.

### DISCLOSURE

### Technical Problem

The disclosure relates to a method of producing FGF21 protein, the method comprising: a) culturing a transformant that produces a FGF21 protein; and b) heating the transformant or culture medium to a temperature of 40° C. or higher.

However, the problem to be solved by the disclosure is not limited to the above-mentioned problem, and other problems not mentioned will be clearly understood by those skilled in the art from the description below.

### Technical Solution

A first aspect of the disclosure provides a method of producing FGF21 protein, the method comprising: a) culturing a transformant that produces a FGF21 protein; and b) heating the transformant or culture medium to a temperature of 40° C. or higher.

A second aspect of the disclosure provides a method of purifying FGF21 protein, the method comprising heating a mixture containing FGF21 protein and impurities at a temperature of 40° C. or higher.

### Advantageous Effects

In the method of producing or purifying the FGF21 protein according to an embodiment of the disclosure, the heat stability and heat-elasticity of FGF21 protein that does not denature or precipitate even after heat treatment and returns to the original structure when re-cooled is used, resulting in advantages of improving efficiency by simplifying a protein production process.

### DESCRIPTION OF DRAWINGS

FIG. 1 is a diagram showing information of the amino acid sequences of an FGF21 protein.

FIG. 2 is a diagram showing results of SDS-PAGE and MASS analysis of the supernatant portion after heat treatment of an FGF21 protein.

FIG. 3 is a diagram showing SDS-PAGE results after heat treatment of a FGF21 terminal-truncation mutant (1) at various temperatures.

FIG. 4 is a diagram showing SDS-PAGE results after heat treatment of an FGF21 wild-type and an FGF21 terminal-truncation mutant @ at various temperatures.

FIG. 5 is a view showing results of UV-visible spectroscopy after heat treatment of an FGF21 protein.

FIG. 6 is a diagram showing aggregation index results after heat treatment and re-cooling of an FGF21 protein.

FIG. 7 is a diagram showing results of CD spectrum analysis of Far-UV region of an FGF21 protein.

FIG. 8 is a diagram showing results of CD spectrum analysis of Near-UV region of an FGF21 protein.

FIG. 9 is a diagram showing SDS-PAGE results after heat treatment of Trx-FGF21.

FIG. 10 is a diagram showing SDS-PAGE results at a purification process of Trx-FGF21 and a process of obtaining a final FGF21 protein.

FIG. 11 is a diagram showing results of an experiment to confirm activation ability of a receptor by FGF21 protein depending on heat treatment.

FIG. 12 is a diagram showing results of an experiment to confirm glucose transport ability by an FGF21 protein in a cell depending on heat treatment.

### BEST MODE

### Mode for Invention

### Examples

### Example 1: Confirmation of Heat-Elasticity of FGF21

Recently, a specific reaction to heat of fibroblast growth factor 21 (FGF21), which is widely known as a protein drug candidate, was discovered, and an FGF21 purification technology was developed using this reaction. In the case of general proteins, a denaturation phenomenon in which tertiary structures of proteins are unfolded is observed at a temperature above certain temperatures. Denatured proteins not only lose the activity, but also aggregate and precipitate. However, in the case of FGF21, it was observed that denaturation and precipitation did not occur even at a high temperature of about 100° C. In addition, it was observed that the structure of FGF21 continuously changes as the temperature increases, but returns to the original structure upon re-cooling and maintains the physiological activity.

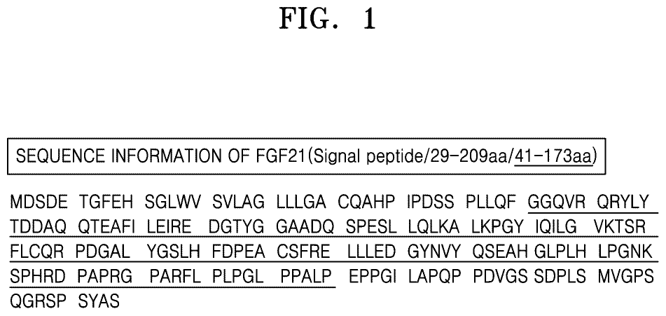

In particular, it was confirmed that these characteristics are maintained as long as the β-trefoil body structure (41-173aa) is preserved, even when some of the N-terminal or C-terminal sequences are removed from wild-type FGF21 (29-209aa) in the mature form. Such maintenance was confirmed on the basis of the structure modeled through the secondary and tertiary structure prediction programs and through the fact that the biochemical and physiological properties of the wild type were experimentally the same as those of terminal-truncated variants. The sequence information of FGF21 is shown in FIG. 1 and Table 1 below. In addition, the specific heat stability/heat reactivity of FGF21 was named heat-elasticity.

TABLE 1

| Name | | | | Sequence | SEQ ID NO. |
|---|---|---|---|---|---|
| Human | Wild type, mature type | | | 29-209 | 1 |
| FGF21 | Terminal- | \textcircled{1} | ΔN12ΔC36 | 41-173 | 2 |
| | truncated | \textcircled{2} | ΔN4 | 33-209 | 3 |
| | variant | | | | |

To confirm the heat stability of the FGF21 protein, the following experiment was performed to observe whether denaturation or precipitation occurs after heat was applied to FGF21, which has been already purified to a high purity of 90% or more at room temperature without heat treatment.

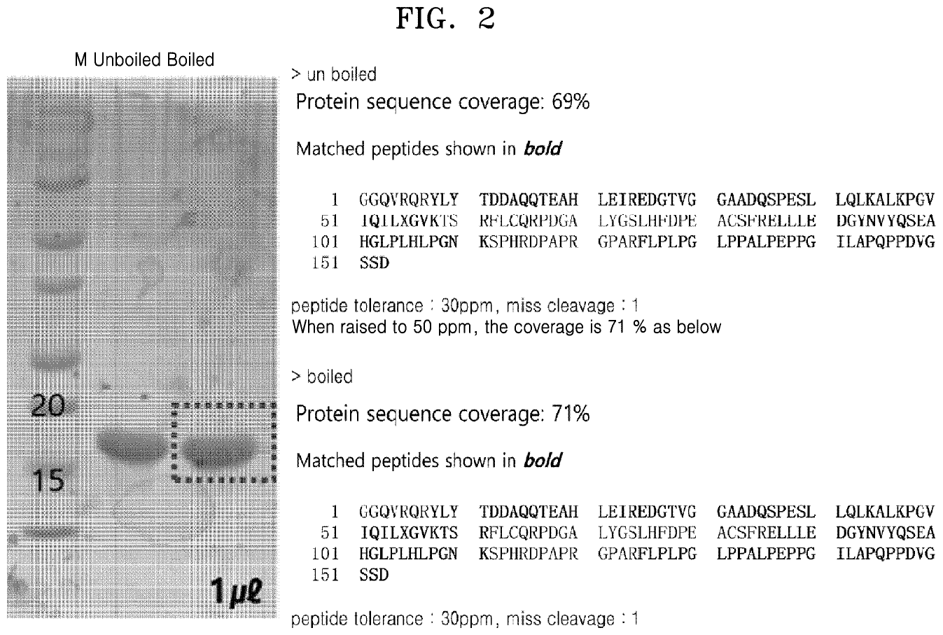

First, the FGF21 (15 mg/ml, 41-192aa) purified to a high purity of 90% or more at room temperature without heat treatment was incubated at 100° C. for 1 hour, followed by centrifugation. After separation into a supernatant fraction and a pellet fraction, SDS-PAGE analysis was performed on each fraction. As a result, FGF21 was found only in the supernatant fraction, and the protein of the corresponding gel was identified to be FGF21 by mass analysis on the corresponding gel (FIG. 2). Referring to the results above, it was confirmed that denaturation and precipitation did not occur in the FGF21 protein even after heat treatment.

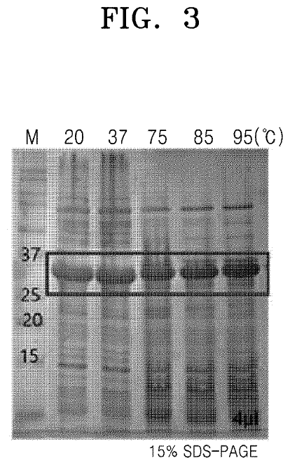

Next, the terminal-truncated mutant n, which is expected to form a β-trefoil structure, was expressed through an E. coli system in the form of a heat-resistant Trx-tag fusion. After cell lysis, a supernatant fraction separated by centrifugation was incubated for 10 minutes at 5 different temperatures. To separate water-insoluble proteins denatured and precipitated by heat treatment, centrifugation (at 14.000 rpm for 20 minutes) was performed again, and as a result of identifying a supernatant fraction thus obtained by SDS-PAGE, it was confirmed that the terminal-truncated variant n exists in a water-soluble state in spite of strong heat treatment at 95° C. (FIG. 3).

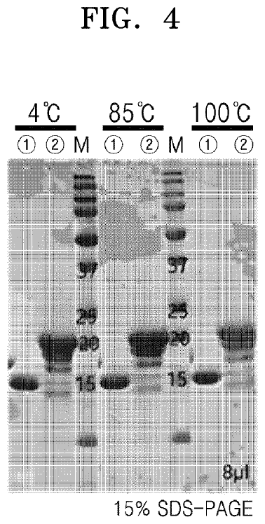

In addition, the variant \textcircled{1}, which is expected to have a β-trefoil main body structure (41-173aa), and an active terminal-truncated variant \textcircled{2}, which includes an interaction site between a receptor and a cofactor, were purified without heat treatment and then incubated at 85° C. and 100° C. for 10 minutes. As a result of SDS-PAGE analysis of the supernatant obtained by centrifugation after the heat treatment, it was confirmed that the band was similar to the band of proteins stored in ice before heat treatment (FIG. 4), and that in the case of having the β-trefoil body structure of FGF21 (41-173aa), the proteins did not precipitate even after heat treatment.

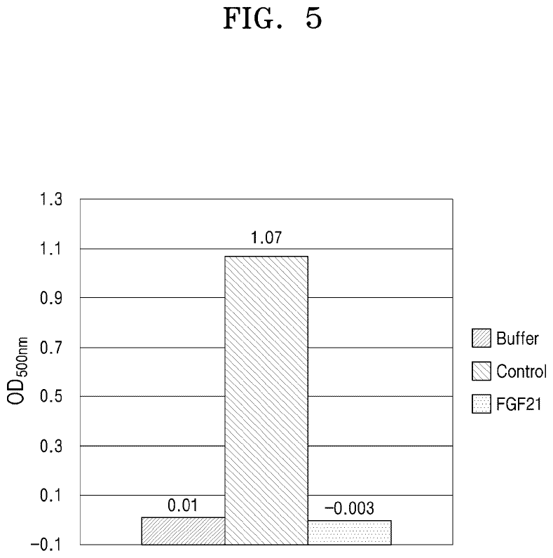

Next, the heat stability of the FGF21 protein was confirmed using a UV-Visible spectrometer. Specifically, when an OD₅₀₀ of a solution containing the FGF21 (15 mg/ml, 41-173aa) that has been purified without heat treatment was measured after incubating the solution at 75° C. for 1 minute, it was confirmed that there was no change in the absorbance value (FIG. 5). Considering that the OD₅₀₀ value of the control, which is a protein easily denatured by heat, increases when precipitation occurs at 75° C., it was confirmed that no precipitation occurred at 75° C. in the case of FGF21.

In addition, in the UV region (240 nm to 380 nm), the aggregation index

$$( A . I . = ( \frac{{OD}_{350 nm}}{{OD}_{280 nm} - {OD}_{350 nm}} ) \times 100 )$$

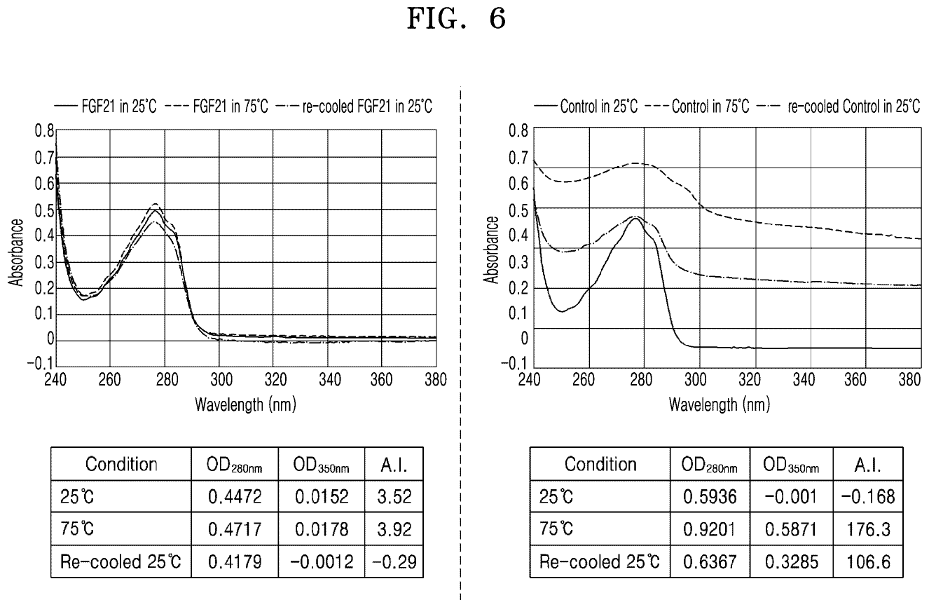

was measured under three conditions (active state at 25° C./state at 75° C./re-cooling state after 1-hour heat treatment at 75° C.) for the FGF21 (15 mg/ml, 41-173aa) and the control protein solution vulnerable to heat. In the case of FGF21 having heat-elasticity, it was confirmed that the change in A.I. value was small and the graph patterns were very similar in the three conditions despite the heat treatment. Meanwhile, in the case of the control protein vulnerable to heat, the A.I. value was calculated as a relatively very high value of 176.3 at 75° C., and was measured as 106.6 even in a re-cooling state. In addition, it was confirmed that the graph pattern of the control protein was greatly changed by heat treatment and was not recovered even after re-cooling (FIG. 6). Based on these results, it was confirmed that the FGF21 has ‘heat-elasticity’ referring that it is denatured by heat treatment and recovers to an active structure upon re-cooling.

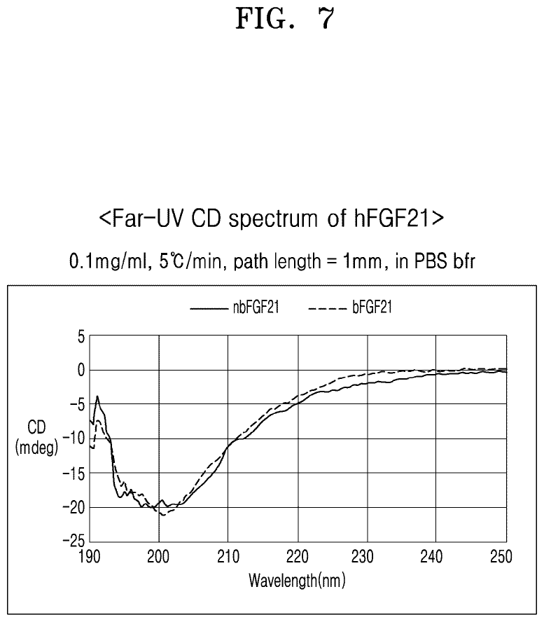

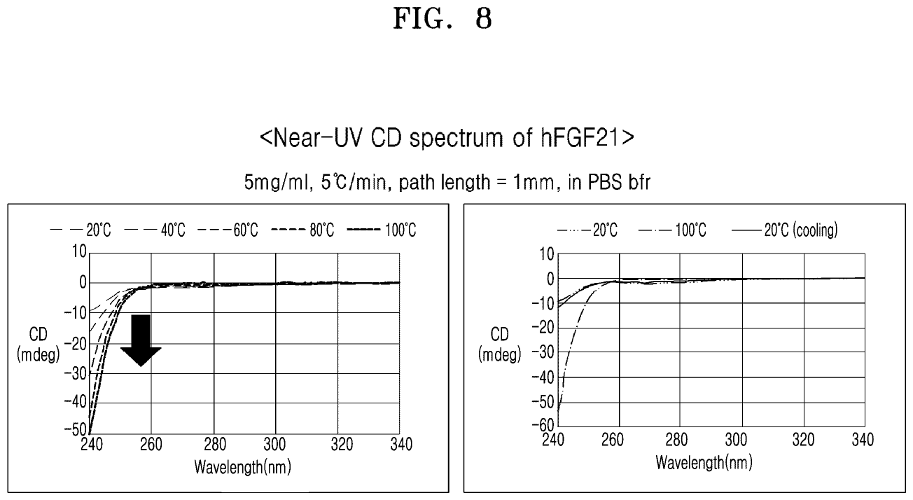

The heat-elasticity of FGF21 was also confirmed in a circular dichroism (CD) experiment. In the Far-UV region (190 nm to 250 nm), which has a unique profile according to the secondary structure ratio of the protein, and in the Near-UV region (240 nm to 340 nm), which is sensitive to the structural change of the protein, changes in the CD spectrum of FGF21 according to temperatures were observed. Specifically, to confirm effects of the heat treatment on the structural changes of FGF21, FGF21 purified without heat treatment (i.e., non-boiled FGF21=nbFGF21, 29-209aa) and purified FGF21 after heat treatment (i.e., boiled FGF21=bFGF21, 33-209aa) were analyzed for the CD spectrum in the Far-UV region. Here, it was confirmed that the ratio of the secondary structure was maintained despite the heat treatment (at 100° C. for 10 minutes) based on the similarity of the CD spectra of nbFGF21 and bFGF21 (FIG. 7). In addition, the CD spectrum of bFGF21 in the near-UV region continuously changes as the temperature rises from 20° C. to 100° C., but after incubation at 100° C. for 5 minutes, the spectrum recovers to the pre-heat treatment spectrum as the temperature decreases to 20° C., thereby confirming the heat-elasticity (FIG. 8).

Summarizing the above results, it was confirmed that the FGF21 protein is not denatured even by performing heat treatment thereon, does not precipitate, and returns to the original structure when re-cooled after heat treatment, indicating that the FGF21 protein has excellent heat stability and heat-elasticity.

### Example 2: Purification Method Using Heat-Elasticity of FGF21

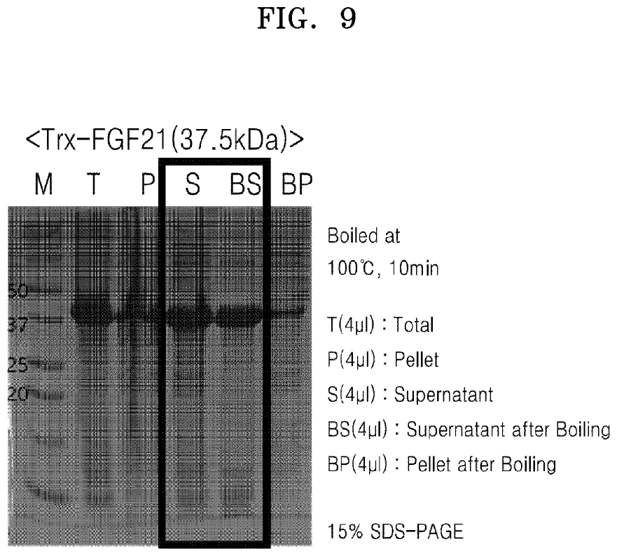

When the heat-elasticity of FGF21 confirmed in Example 1 is used in a protein purification process, a production process can be simplified and a production cost can be reduced. First, to increase the expression level of FGF21, a heat-resistant protein, such as thioredoxin (Trx) or DnaK, may be used as a tag. FGF21 fused with such a heat-resistant protein can be overexpressed in an E. coli system and purified through various chromatographic techniques. However, by introducing a heat treatment process, the protein purification process can be simplified. In detail, to purify/secure high-purity recombinant FGF21 (33-209aa) by using the heat-elasticity of FGF21, E. coli in which Trx-FGF21 consisting of FGF21 fused to Trx was overexpressed was disrupted by sonication, followed by centrifugation. The supernatant solution containing Trx-FGF21 was incubated at a high temperature (50° C. to 100° C.) for 10 minutes, and then proteins denatured at a high temperature and Trx-FGF21 were separated by centrifugation. Through SDS-PAGE analysis of samples at each stage, it was confirmed that Trx-FGF21 was present in a water-soluble state (FIG. 9).

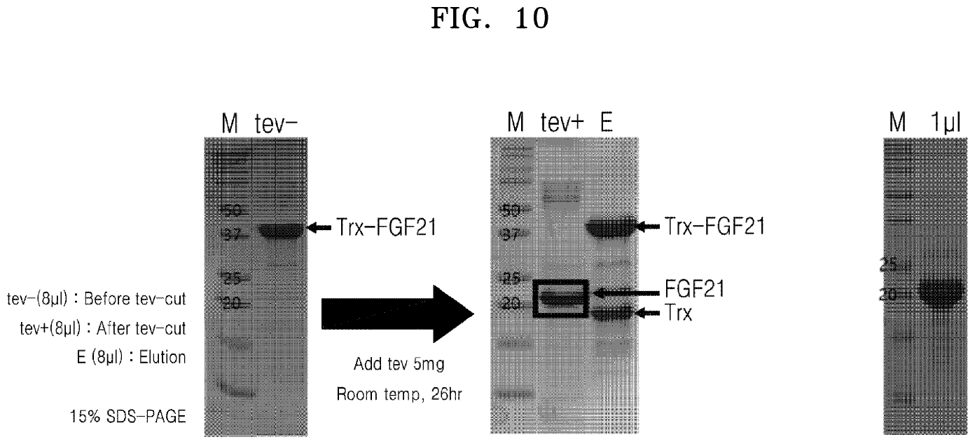

In addition, to secure pure FGF21 from which Trx was removed, a cleavable linker must be inserted between the tag and FGF21. Specific sequences recognized by a tobacco etch virus (TEV)-derived protease or a protease such as thrombin and Factor Xa, etc. can be used as a linker, and it was confirmed that FGF21 can be isolated by using such linkers (FIG. 10). Therefore, FGF21 can be separated from the tag by a protease, and then purified with a high purity of 90% or more by using two or less chromatographic techniques. In addition, when an asparagine-glycine (Asn-Gly) linker is inserted between the tag and FGF21, the linker can be cleaved by heat treatment (40° C. to 42° C., 3 hours) by using hydroxylamine (NH₂OH).

Taken together, a protein purification process may include a heat treatment process when the excellent heat stability and elasticity of FGF21 are utilized, and accordingly, processes of removing impurities and other proteins can be simplified. By fusion with a heat-resistant protein tag to FGF21, there is an advantage of improving a production yield and the like.

### Example 3: Confirmation of Physiological Activity of Heat-Treated FGF21

FGF21 exhibits various physiological effects by activating fibroblast growth factor receptor 1c isoform (FGFR1c), which is one of FGF receptors. Therefore, to confirm an effect of heat treatment on the activity of FGF21, the following experiment was performed.

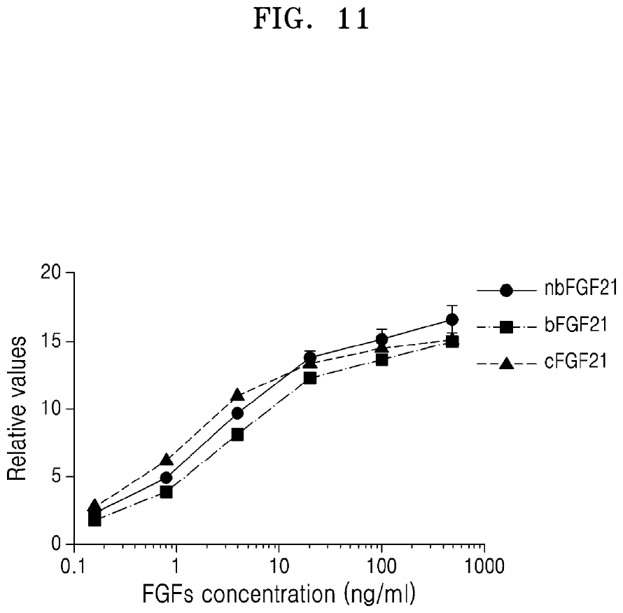

In detail, the FGFR1c signaling ability of nbFGF21 (33-209aa), bFGF21 (33-209aa), and commercially available FGF21 (commercial FGF21=cFGF21, 29-209aa) was confirmed. For this, modified HEK293 cells (iLite FGF21 assay ready cells, Euro Diagnostica, Cat #BM3071) expressing FGFR1c and FGF receptor-binding cofactor, ß-Klotho, and having a reporter system capable of confirming the degree of activation of FGFR1c were used. As a result of the experiment, it was confirmed that the receptor activation ability of bFGF21 was similar to that of nbFGF21 and cFGF21 (FIG. 11).

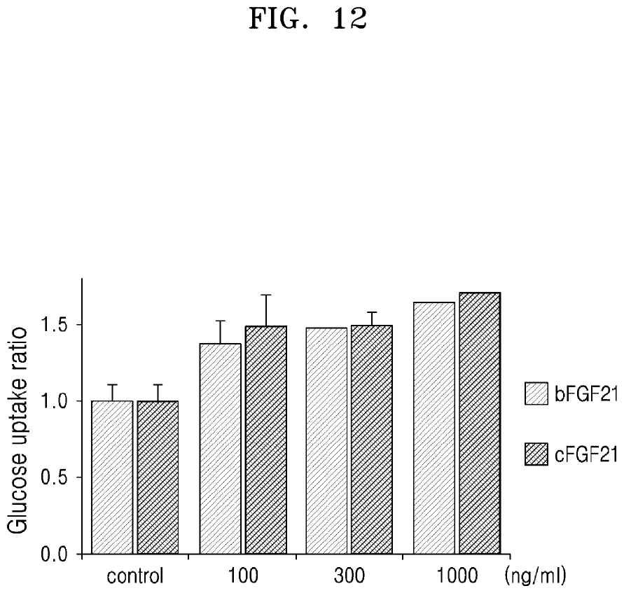

In addition, since FGF21 promotes glucose transport and has an activity to increase cellular glucose uptake, the glucose transport capability of heat-treated FGF21 was confirmed. In detail, to confirm the glucose transport ability, 2-deoxyglucose (2DG) was used. A glucose transporter that transports glucose into cells does not distinguish between glucose and 2DG, and 2DG absorbed into cells is phosphorylated in the same way as glucose to form 2-deoxyglucose-6-phosphate (2DG6P), but cannot proceed to the next step anymore. Thus, the degree of glucose transport ability can be confirmed by measuring the amount of 2DG6P. By using glucose Uptake-Glo assay (Promega, Cat #J1342), a which is product for confirming the amount of 2DG6P in cells using a luminometric method, and 3T3L1 adipocytes, which are mainly expressed by FGFR1c to which FGF21 binds, the glucose transport ability of FGF21 proteins was measured. As a result, it was confirmed that the activity of bFGF21 was similar to that of cFGF21 (FIG. 12).

Taken together, it was confirmed that, when the FGF21 protein was heat treated or re-cooled after heat treatment, the physiological activity of the FGF21 protein was the same/similar to that before heat treatment, and thus even when the FGF21 protein was produced/purified through a heat treatment process, there is no abnormality in the function of the FGF21 protein.

The foregoing descriptions are only for illustrating the disclosure, and it will be apparent to a person having ordinary skill in the art to which the disclosure pertains that the embodiments disclosed herein can be easily modified into other specific forms without changing the technical spirit or essential features. Therefore, it should be understood that Examples described herein are illustrative in all respects and are not limited. For example, each component described as in a single type may be implemented in a distributed manner, and likewise components described as being distributed may be implemented in a combined form.

The scope of the disclosure is indicated by the following claims rather than the detailed description above, and all changes or modifications derived from the meaning and scope of the claims and equivalent concepts thereof should be construed as being included in the scope of the disclosure.

1. A method of producing an FGF21 protein, the method comprising:
a) culturing a transformant producing an FGF21 protein; and
b) heating the transformant or culture medium at a temperature of 40° C. or higher.

2. The method of claim 1, wherein
the FGF21 protein comprises a wild type or a variant, and
the wild type or variant comprises the amino acid sequence of SEQ ID NO: 2.

3. The method of claim 1, wherein the FGF21 protein is has heat stability or heat-elasticity.

4. The method of claim 1, wherein the FGF21 protein has a β-trefoil structure.

5. The method of claim 1, wherein the FGF21 protein is additionally fuse with a thermostable protein tag.

6. The method of claim 1, further comprises c) removing proteins denatured by heat treatment.

7. A method of purifying an FGF21 protein, the method comprising heating a mixture containing an FGF21 protein and impurities at a temperature of 40° C. or higher.

8. The method of claim 7, further comprising: removing proteins denatured by heat treatment.


SEQUENCE LISTING (3 sequences)

SEQ ID NO: 1 - protein, 181 aa, Artificial Sequence
  human FGF_wild type, mature form
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 133 aa, Artificial Sequence
  human FGF_N12C26
GGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPA

SEQ ID NO: 3 - protein, 177 aa, Artificial Sequence
  human FGF_N4
DSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS